(19) United States
(12) Patent Application Publication
(10) Pub. No.: US 2006/0172314 A1
(43) Pub. Date: Aug. 3, 2006

(54) Quantification of amplified nucleic acids

(76) Inventors: Min-sun Song, San Francisco, CA (US); Rhodora Argonza-Barrett, Mountain View, CA (US); Diane D. Ilsley, San Jose, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 11/047,385

(22) Filed: Jan. 31, 2005

(51) Int. Cl. C12Q 1/68; C12P 19/34

(52) U.S. Cl. 435/6; 435/91.2

(57) ABSTRACT

The invention relates to methods and kits for quantification of amplified nucleic acids, such as genomic DNA. The methods and kits can be used to assess bias in an amplification procedure such as a whole genome amplification procedure. In one aspect, the method comprises performing an enzyme-based amplification procedure and validating the results of the procedure using a signal amplification method.

### BACKGROUND

The identification of differences in gene dosage or expression among cell populations is important for the study and detection of disease. For example, cancer is typically associated with acquired genomic instability and the evolution of neoplastic cell lineages that develop multiple copy number variations (losses and/or gains) of genomic DNA. The gain of DNA sequences can be correlated with the activation of oncogenes, while the loss of DNA sequences can be correlated with the inactivation of tumor suppressor genes. Thus, the identification of the genetic events leading to neoplastic transformation and subsequent progression can facilitate efforts to define the biological basis for disease, improve prognosis and permit earlier cancer detection.

Comparative genomic hybridization (CGH) is one approach that has been employed to detect the presence and identify the location of amplified or deleted DNA sequences on a genome-wide basis. In one application of CGH, genomic DNA is isolated from normal reference cells as well as from test cells, e.g., such as tumor cells. Reference and test nucleic acids are differentially labeled and then simultaneously hybridized in situ to metaphase chromosomes of a reference cell. Chromosomal regions in the test cells having an increased or decreased copy number can be identified by detecting regions where the ratio of signal from reference and test DNA is altered. See, e.g., Kallioniemi et al., Science 1992; 258(5083):818-21; Pollack et al., Proc Natl Acad Sci USA 2002;99(2):12963-8.

Because the progression of cancer in an organism results in multiple genetically distinct populations being present even in small samples, solid tumor and premalignant tissue samples frequently contain a mixture of neoplastic cells as well as a variety of normal cell types such as infiltrating lymphocytes, stromal cells and surrounding non-tumor epithelial cells. The presence of normal cells in a tumor sample can obscure the measurement and detection of genomic changes while the variable genomic lesions present in multiple neoplastic cell populations cannot be resolved without prior clonal purification.

Techniques to purify cell populations of interest from clinical and biological samples include laser capture microdissection and flow cytometry. These typically result in the isolation of small samples that become highly valuable due to the effort to prepare them and their potential for biological and clinical information content. However, patient care and management (e.g., pathological staging) often requires relatively large amounts of material from clinical samples of interest. Furthermore, additional materials from these same samples may be needed for archiving, follow-up studies, as well as repeat and validation experiments.

Existing protocols for comprehensive genomic scanning of tumor samples typically require use of either an entire valuable sample, multiple preparations, or the use of mixed (e.g., normal and neoplastic cells) samples. For example, current practice typically requires up to 20 μg of genomic DNA (gDNA) as starting material for array-based CGH (aCGH) (Pollack J R et al, 2002, supra; Hyman E. et al, Cancer Res. 2002;62(21):6240-5). All of these requirements make it difficult to apply CGH techniques in clinical settings. There is a significant need for a reliable whole genomic amplification (WGA) method to supply sufficient quantities of genomic sequence for an ever-growing number of genetic tests like CGH. Although several methods are available for generating nucleic acids representing whole genomes from small samples, comprehensive genome scanning requires accurate and efficient amplification of starting materials.

The phi29 DNA polymerase can be used to perform unbiased amplification across the human genome, with relative representation of individual loci differing by less than 6-fold compared to unamplified genomic DNA (Hosono et al, Genome Res. 2003; 13(5):954-64). This permits both reference and test DNA samples to be amplified prior to inclusion in an aCGH experiment. Multiple displacement amplification (MDA) relying on the phi29 enzyme is an isothermal amplification method which exploits the highly processive nature of phi29 to polymerize>70 kb without dissociating from a genomic DNA template. This DNA polymerase has a 3′ to 5′ exonuclease proofreading activity to maintain high fidelity replication and is used in the presence of exonuclease-resistant primers to achieve high yields of DNA product from small starting amounts of clinical samples.

It is crucial to quantitate the product generated by phi29 DNA polymerase or other enzymes used for whole genome amplification, since aCGH analysis relies on the assumption that the amount of test and reference nucleic acids applied to a probe array accurately reflects the relative amounts of genomic DNA found in test and reference cell samples. However, the multiple strand displacement activity of phi29 DNA polymerase can cause a high level of branched nucleic acid forms from a degraded DNA sample, resulting in non-uniform amplification. Furthermore, the use of phi29 polymerase with degraded samples can result in low or insufficient yields of high molecular weight DNA suitable for downstream applications such as fluorescence labeling (Molecular Staging Inc., User Manual, New Haven, Conn.). To ensure complete coverage and representation of the genome to be amplified, current protocols using phi29 polymerase typically require high quality intact genomic DNA as a starting material (Pollack J R et al, 2002, supra; Hyman E, et al, 2002, supra). In addition, the presence of high concentrations of primers and the production of variable amounts of primer-specific amplification products in the phi29 reaction make it difficult to determine the yield of high molecular weight DNA in an amplification reaction by methods such as UV/vis spectroscopy.

Several methods have been used to validate the amplified target by phi29 DNA polymerase which include quantitative PCR analysis. However, reliance on PCR applications makes the testing of multiple samples difficult and increases the risk of carry-over contamination with amplified PCR products.

### SUMMARY

In one embodiment, the invention relates to a method for quantitating nucleic acid samples. Methods according to the invention can be used to quantitate amplified nucleic acid targets, such as genomic nucleic acids. In one embodiment, the method is used to quantitate products of an enzyme-based whole genomic amplification technique or an unbiased or partially biased amplification technique and comprises obtaining a portion of an amplified sample, prior to or after amplification and quantitating one or more sequences in that portion of the sample by a non-enzyme based amplification technique. In one aspect, the non-enzyme based amplification method is one that relies on signal amplification, e.g., the method comprises contacting nucleic acids in the portion of the sample with a probe set comprising multi-labeled probe sequences. The probe sequences can be labeled directly or indirectly with multiple labels (e.g., using linear or branched multimeric DNA molecules).

In one embodiment, the invention relates to a method comprising performing an enzyme-based amplification method on a sample comprising nucleic acids to obtain amplified nucleic acids; obtaining a first and second portion of sample comprising the amplified nucleic acids; contacting amplified nucleic acids in the first portion to a probe set comprising a multi-labeled probe nucleic acid molecule comprising a probe sequence, wherein the multi-labeled probe nucleic acid molecule comprises a plurality of labels;

comparing a level of nucleic acids complementary to the probe sequence in the second portion to a level of nucleic acids bound to the multi-labeled probe nucleic acid molecule in the first portion.

In one aspect, the nucleic acids comprise genomic DNA and the enzyme-based amplification method comprises a whole genome amplification method. In another aspect, the enzyme-based amplification method comprises contacting sample nucleic acids with random primers or degenerate primers in the presence of nucleotides and a polymerase. In certain aspects, linkers comprising constant sequences are ligated to the ends of fragmented sample nucleic acids (e.g., such as generated by exposing the sample to shearing conditions) and the nucleic acids are contacted with primers complementary to the linkers. In a further aspect, the polymerase is a phi29-like polymerase.

In one embodiment, the multi-labeled probe nucleic acid molecule comprises a multimeric nucleic acid molecule comprising a plurality of repeating sequence units and a probe sequence or a complement of a probe sequence.

In another embodiment, the method comprises obtaining a portion of the sample prior to amplification and contacting unamplified nucleic acids in the portion to a probe set comprising a multi-labeled probe nucleic acid molecule comprising a probe sequence, wherein the multi-labeled probe nucleic acid molecule comprises a plurality of labels. In one aspect, the method further comprises comparing a level of nucleic acids complementary to the probe set in the sample comprising amplified nucleic acids to a level of nucleic acids in the portion of the sample comprising unamplified nucleic acids which are bound to the probe sequence in the probe set.

In certain aspects, the probe set nucleic acid molecules comprise a plurality of different probe sequences. In one aspect, the probe set comprises at least about 5 different nucleic acid sequences. In another aspect, the probe set comprises at least about 10 different probe sequences. In still another aspect, when the probe set comprises a plurality of multi-labeled probe nucleic acid molecules, each member of the plurality comprising a different probe sequence, the portion of sample to be contacted with the probe set is divided into a number of portions corresponding to the number of members, for contacting each of the divided portions with a different member of the plurality.

The method can be used to assess an amount of bias in the enzyme-based amplification method. In one aspect, the probe set comprises a plurality of different probe sequences and bias is assessed by determining the numbers of different probe sequences in the probe set that binds to the portion of amplified nucleic acids. In another aspect, the probe set comprises a plurality of different probe sequences and bias in the enzyme-based amplification procedure is assessed by determining the numbers of different probe sequences in the probe set that binds to the portion of unamplified nucleic acids compared to numbers of different probe sequences in the probe set that bind to the portion of amplified nucleic acids. In a further aspect, the probe set comprises a plurality of different probe sequences and the relative proportions of bound probes in the portion of sample comprising amplified sequences is compared to the relative proportions of bound probes in the portion of sample comprising unamplified sequences.

In one embodiment, the method further comprises labeling amplified sequences in a primer extension reaction by providing either, or both, labeled primers and nucleotides, and comparing the amount of sequences complementary to sequences of probes in the probe set to the relative proportions of bound probes in the portion of sample comprising amplified sequences and/or in a portion of sample comprising unamplified sequences.

In another embodiment, the method comprises contacting a first portion of an amplified test nucleic acid sample to an array of probe sequences under specific binding conditions and identifying a first complex formed between a test nucleic acid and a probe sequence on the array. The amount of first complex formed is compared to an amount of a second complex formed between an amplified reference sequence from a reference sample and the same probe sequence and/or the amount of a second complex formed between an amplified reference sequence present at a known level in the amplified test sample (e.g., such as reflecting an amplified diploid amount of the reference sequence) and a different probe sequence. The amount of the first and second complex are compared. For example, the ratio of the amount of the first complex to the second complex can be determined. A second portion of the amplified test genomic nucleic acid sample is contacted under specific binding conditions to a probe set comprising a multi-labeled probe nucleic acid molecule comprising the probe sequence, wherein the multi-labeled probe nucleic acid molecule comprises a plurality of labels. The amount of a third complex, i.e., that formed between the multi-labeled probe nucleic acid molecule and amplified test nucleic acids in the second sample is determined. In one aspect, the amount of the third complex is compared to the amount of the first and/or second complex. In another aspect, the amount of the third complex is compared to the amount of a fourth complex formed between a reference nucleic acid molecule from a reference sample and the same multi-labeled probe nucleic acid molecule or between a reference nucleic acid molecule present at a known level in the test sample and a different multi-labeled probe nucleic acid molecule. In a further aspect, the ratio of the amount of first complex to the amount of second complex is compared to the ratio of the amount of third complex to the amount of fourth complex.

In another embodiment, a sample of test nucleic acid is obtained prior to its amplification and a portion of the sample is contacted with the multi-labeled probe nucleic acid molecule under specific binding conditions and the amount of a fifth complex formed between the multi-labeled probe nucleic acid molecule and test nucleic acid is determined. The amount of the fifth complex can be compared to amounts of the first, second, third, and/or fourth complex and/or to a sixth complex formed between a portion of an unamplified reference nucleic acid from a reference sample and the same multi-labeled probe or a reference nucleic acid within the portion of the unamplified test nucleic acid sample and a different multi-label probe (e.g., complementary to the reference nucleic acid).

In one embodiment, the amplified test nucleic acids are labeled. In one aspect, the amplified test nucleic acids are labeled after removing a portion of the amplified test nucleic acid sample for contacting with the probe set comprising the multi-labeled probe. In another aspect, the amplified test nucleic acids are labeled during amplification but after removing a portion of unamplified sample for contacting with the probe set.

In certain aspects, complexes are distinguished from each other through the use of distinguishable labels, such as spectrally distinguishable labels. In other aspects, complexes are distinguishable from each other by where they are formed, e.g., on one location of a substrate (e.g., an array or support comprising a capture probe) vs. another.

In certain embodiments, e.g., where the amplified reference nucleic acids are from a reference sample, the amplified reference nucleic acids are labeled during or after amplification, with a label which is distinguishable from the label used to label amplified test nucleic acids applied to the array.

In one embodiment, the test sample and reference sample comprises genomic nucleic acids such as genomic DNA. In one embodiment, the test sample and reference sample comprise amplification products produced from a non-biased enzyme-based amplification process, e.g., using primers that bind to sequences present randomly in the nucleic acid molecules of the sample or which are present abundantly (e.g., such as linker sequences added to genomic fragments after a ligation reaction).

In certain aspects, the selection of probe sequences which form the probe set is based upon the formation of first complexes on the array. In one aspect, a probe sequence for the probe set is selected which forms a desired predetermined amount of first complex with an amplified test genomic sample (e.g., such as a labeled amplified test genomic sample). For example, in one aspect, the desired predetermined amount of complex is at least about three standard deviations different from a background amount (such as an amount of test sample which binds to an inter-feature area on the array or to a non-complementary probe). In another aspect, the probe set comprises a plurality of probe sequences. The probe sequences can represent a range of amounts of first complexes observed upon the array, from a background level, to a level significantly above background, to a level approaching saturation for the detection system used to detect the label being used to label test sequences applied to the array. In one aspect, the method comprises identifying a probe sequence on the array for which the ratio of first and second complexes is different from one, and including the probe sequence in the probe set. In another aspect, the method comprises identifying a probe sequence on the array for which the ratio of first and second complexes is one and including the probe sequence in the probe set.

In one embodiment, the nucleic acids in the second portion of the amplified test genomic nucleic acid sample are stably associated with a solid support. In one aspect, the solid support comprises capture oligonucleotides, which hybridize to a portion of the amplified test genomic nucleic acids. In certain aspects, the amplified test genomic nucleic acid samples are ligated to sequences complementary to at least a portion of the capture oligonucleotides or are amplified with primers which include both sequence selective regions for hybridizing to the genomic DNA and constant regions which are designed to be complementary to the capture oligonucleotide sequences and can be used to capture amplified test genomic nucleic acids.

In another embodiment, the capture oligonucleotides are selected to selectively capture only a portion of the amplified test genomic DNA, e.g., that portion comprising sequences complementary to probe sequences in the probe set. Thus, in certain embodiments, a capture oligonucleotide is selected to hybridize to a genomic sequence which comprises a different sequence from the sequence which is complementary to the probe sequence. In one aspect, the capture oligonucleotide sequence is complementary to a sequence found with a high representation throughout the genome (e.g., such as an Alu sequence).

In one aspect, the plurality of label molecules are attached to a probe sequence of the multi-labeled nucleic acid molecule by hybridizing a multimeric nucleic acid sequence comprising a sequence complementary to a portion of the probe sequence and a plurality of repeating sequence units which can each bind, directly or indirectly, to a label molecule. The multimeric nucleic acid can be linear or branched. In one aspect of the invention, the multimeric nucleic acid is branched and in certain aspects, a plurality of branches of the branched nucleic acid molecule bind, directly or indirectly to a label molecule. In one aspect, the label molecule comprises a nucleic acid molecule comprising a detectable molecule conjugated thereto. The label molecule can be directly or indirectly detectable. For example, the label molecule can comprise a detectable molecule that is an enzyme (e.g., such as alkaline phosphatase) and the amount of label can be detected by detecting a reaction catalyzed by the enzyme.

In one embodiment, the probe set comprises a plurality of different multi-labeled nucleic acid molecules comprising different probe sequences. In one aspect, the probe set comprises at least about 5 different probe sequences. In another aspect, the probe set comprises at least about 10 different probe sequences. The number of different probe sequences in a probe set can be varied depending on the desired resolution of the assay. In one aspect, probe sequences are selected which bind to sequences with separated by at least about 300 bp, at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. The resolution of the probe set can be the same or different as the resolution of probes on the array. In a further aspect, at least two different probe sequences form different amounts of complexes on the array. In one aspect, the different probe sequences form complexes which differ by an at least one-fold amount. In one aspect, the probe set comprises a sequence complementary to at least one “housekeeping gene,” e.g., such as actin or tubulin. In another aspect, the probe set is biased to include sequences complementary to sequences whose copy number changes have been associated with a disease (e.g., such as breast cancer).

Methods for amplifying test sequences prior to contacting to the array can vary. In one aspect, genomic nucleic acid is amplified using an isothermal amplification technique. In another aspect, nucleic acid is amplified using a strand displacement technique, such as multiple strand displacement. In a further aspect, the nucleic acid is amplified using random primers. In certain aspects of the invention, methods according to the invention can be used to validate lack of bias in an amplification protocol, such as a whole genome amplification protocol. In one embodiment, the genomic nucleic acid is amplified using a phi29-like enzyme. In another embodiment, the genomic nucleic acid is amplified using a helicase.

In one embodiment, the ratio of first to second complexes is used to determine the copy number of a genomic nucleic acid in a sample. Copy number determination can include the determination of both gain and loss of sequences. In a one aspect, copy number determination is correlated with one or more characteristics of a patient supplying a sample, e.g., such as a disease. In another aspect, copy number determination is associated with changes in characteristics of a patient, e.g., comparing samples of nucleic acids from a patient obtained at different time periods.

In still another aspect, copy number determination is used to screen samples to identify patients at risk for a disease associated with a genomic imbalance such as cancer. In still another aspect, copy number determination is used to screen samples to determine prognosis of a disease associated with a chromosomal imbalance such as cancer. In a further embodiment, the invention is used in prenatal testing and/or to detect chromosomal abnormalities in somatic and/or germline cells.

In certain aspects, the array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. In certain aspects, the array comprises both non-coding and coding sequences. In one aspect, individual probes on the array comprise either coding or non-coding sequences but do not comprise both coding and non-coding sequences. In certain aspects, individual probes comprise sequences that do not normally occur together, e.g., to detect gene rearrangements, for example.

In another aspect, while amplification of a nucleic acid sample is unbiased, the complexity of the sample is reduced compared to a cell from which the nucleic acid is obtained. For example, in one aspect, prior to amplification, sample nucleic acid sequences (genomic sequences or RNA sequences) are selected by their ability to bind to one or more nucleic acid binding proteins. In certain aspects, the sequences which bind to the one or more nucleic acid binding proteins are then amplified using a non-biased amplification method (e.g., such as MDA) and applied to the array. Complexes formed between the amplified sequences and probes on the array are used to identify the types and/or genomic locations of sequences, which were bound to the nucleic acid binding proteins. In certain aspects, nucleic acid sequences bound to nucleic acid binding proteins are cross-linked to the proteins and cleaved with a cleavage agent to remove or decrease the amount of nucleic acid sequences outside of the binding region to which the proteins are bound. Bound (and optionally crosslinked complexes) can be removed from non-bound nucleic acids and amplified and the amplified nucleic acids corresponding to these binding regions are then applied to the array. As above, methods according to the invention can be used to quantify selected sequences in the amplified sample and/or can be used to validate the lack of bias in the amplification step.

Test samples can be obtained from a variety of sample sources. In one aspect, the test sample is from a biopsy. In another aspect, the test sample is from a tumor. In still another aspect, the sample is from a source of fetal nucleic acids, such as amniotic fluid or chorionic villus cells. The source of reference nucleic acid also can vary. In one aspect, the reference nucleic acid is from a healthy patient. In another aspect, the reference nucleic acid is from an individual known to have a diploid complement of a nucleic acid or a known amount of the nucleic acid. In certain aspects, the reference nucleic acid is from the test sample.

The invention also relates to kits for facilitating the above methods. In one aspect, the invention provides a kit comprising a probe set for use in a method according to the invention. In another aspect, the kit comprises an array for use in a method according to the invention. In a further aspect, the kit comprises both an array and a probe set according to the invention. In still another aspect, the invention provides a kit comprising reagents for performing an enzyme-based amplification method and a non-enzyme based amplification method, such as a signal amplification method. In one aspect, the kit comprises a probe set comprising a multi-label nucleic acid molecule and a polymerase. In another aspect, the kit comprises a phi29-like enzyme and/or a branched nucleic acid molecule. In still another aspect, the kit comprises a probe set comprising a multi-label nucleic acid molecule and random primers. In a further aspect, the kit includes a multi-well plate (e.g., such as a microtiter plate) comprising capture oligonucleotides stably associated with a least a portion of a plurality of the wells. The capture probes can be used to immobilize sample nucleic acids to be contacted with a probe set. In certain aspects, the multi-well plates comprise at least one array at the base of a well. In certain aspects, the multi-well plates comprise both arrays and capture probes in different wells. In certain aspects, the probe set comprises a plurality of multi-labeled nucleic acid molecules, each member of the plurality comprising a different probe sequence, and each member is provided in a different container or in different wells of a container with a plurality of wells.

### BRIEF DESCRIPTION OF THE FIGURES

The objects and features of the invention can be better understood with reference to the following detailed description and accompanying drawing.

FIGS. 1A and B are flow charts illustrating methods according to embodiments of the invention.

### DETAILED DESCRIPTION OF THE INVENTION

Before describing the present invention in detail, it is to be understood that this invention is not limited to specific compositions, method steps, or equipment, as such may vary. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless defined otherwise below, all technical and scientific terms used herein have the same meaning as commonly understood by one of ordinary skill in the art to which this invention belongs. Still, certain elements are defined herein for the sake of clarity.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that, as used in the specification and the appended claims, the singular forms “a,” “an” and “the” include plural referents unless the context clearly dictates otherwise. Thus, for example, reference to “a nucleic acid molecule” includes a plurality of nucleic acid molecule, reference to “a probe” includes a mixture of probes, and reference to “a characteristic” includes a plurality of characteristics and the like.

The following definitions are provided for specific terms that are used in the following written description.

Definitions

The term “nucleic acid” as used herein means a polymer composed of nucleotides, e.g., deoxyribonucleotides or ribonucleotides, or compounds produced synthetically (e.g., PNA, LNA, or UNA molecules) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “functionalization” as used herein relates to modification of a solid substrate to provide a plurality of functional groups on the substrate surface. By a “functionalized surface” is meant a substrate surface that has been modified so that a plurality of functional groups are present thereon.

The terms “reactive site”, “reactive functional group” or “reactive group” refer to moieties on a monomer, polymer or substrate surface that may be used as the starting point in a synthetic organic process. This is contrasted to “inert” hydrophilic groups that could also be present on a substrate surface, e.g., hydrophilic sites associated with polyethylene glycol, a polyamide or the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids which are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiments, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

By “genomic source” is meant the initial nucleic acids that are used as the original nucleic acid source from which the probe nucleic acids are produced, e.g., as a template in the nucleic acid amplification and/or labeling protocols described in greater detail below.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide elements employed herein are present on a surface of the same planar support, e.g., in the form of an array. It should be understood that the terms “probe” and “target” are relative terms and that a molecule considered as a probe in certain assays may function as a target in other assays.

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

As used herein a “multi-labeled nucleic acid molecule comprising a probe sequence” refers to a nucleic acid molecule that includes a probe sequence complementary to a target sequence of interest. In one aspect, a probe sequence comprises at least about 6, at least about 8, at least about 10, at least about 15, at least about 20, at least about 25, at least about 30, at least about 40, at least about 50 to about 200 bases. The probe sequence is part of or hybridizes to a multimeric nucleic acid sequence that comprises the probe sequence or its complement and a plurality of repeat sequence units. The repeat units may comprise label molecules or may bind to nucleic acids which themselves bind (directly or indirectly, e.g., through additional nucleic acid molecules) to label molecules. In one aspect, the binding of multi-labeled nucleic acid molecule to a target sequence provides linear signal amplification for the detection of probe:target complexes compared to the exponential amplification obtained from an enzyme-based assay. The terms “multi-labeled nucleic acid molecule” and “multi-labeled nucleic acid composition” can be used interchangeably.

As used herein, the term “stably associated with a solid support” refers to an association with the support that does not substantially change under the conditions in which the support is used (e.g., such as stringent hybridization conditions and washing conditions). A stable association may be generated by covalent, ionic or nonionic associations.

As used herein, a “probe set comprising different probe sequences” refers to a plurality of nucleic acid molecules comprising a sequence or subsequence which specifically binds to a target under stringent hybridization conditions, where each of the probe sequences differs by at least one nucleotide. It should be noted that nucleic acid molecules comprising different probe sequences may each comprise sequences and/or moieties in addition to the probe sequence, such as constant regions, linker regions, repeat sequences, identifier tags, labels and the like, which may be the same or different between nucleic acid molecules. Further, a nucleic acid molecule comprising a probe sequence may comprise more than one molecule.

As used herein, an “enzyme-based amplification method” refers to a method which relies on an enzyme such as a polymerase to increase the quantity of a nucleic acid population.

As used herein, “amplifying conditions” are conditions under which a polymerase will extend a primer sequence which is hybridized to a sequence to be amplified to produce a sequence complementary to the sequence to be amplified.

As used herein, a “multi-well plate comprising an array of nucleic acid probes in at least one well” refers to well comprising an array substrate on which a plurality of nucleic acid probes are arrayed. In one aspect, the substrate is attached to the base of the well, e.g., by gluing (e.g., by ultraviolet-curing epoxy or various sticking tapes), acoustic welding, sealing such as vacuum or suction sealing, by means of pressure or clamping. However, in another aspect, the substrate merely rests at the base of the well and may be suspended in fluid when the array is exposed to a fluid. In still another aspect, the array substrate is the base of the well, e.g., the multi-well plate comprises a base formed by the substrate and a body comprising a top surface and a bottom surface and a plurality of openings from the top surface through to the bottom surface such that placement of the body on the substrate produces the multi-well plate.

The term “array” encompasses the term “microarray” and refers to an ordered array presented for binding to nucleic acids and the like.

An “array,” includes any two-dimensional or substantially two-dimensional (as well as a three-dimensional) arrangement of addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100μ², or even smaller. For example, features may have widths (that is, diameter, for a round spot) in the range from a 10 μm to 1.0 cm. In other embodiments each feature may have a width in the range of about 1.0 μm to 1.0 mm, usually 5.0 μm to 500 μm, 5.0 μm to 100 μm, and 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences which can function as probes) such that a region (i.e., a “feature” or “spot” of the array) at a particular predetermined location (i.e., an “address”) on the array will detect a particular complementary sequence. Array features are typically, but need not be, separated by intervening spaces. In certain aspects, nucleic acid sequences applied to the array to be detected are present in a liquid sample.

As used herein, the term “signal” refers to the detectable characteristic of a detectable molecule. Exemplary detectable characteristics include, but are not limited to: a change in the light adsorption characteristics of a reaction solution resulting from enzymatic action of an enzyme attached to a labeling probe acting on a substrate; the color or change in color of a dye; fluorescence; phosphorescence; radioactivity; or any other indicia that can be detected and/or quantified by a detection system being used.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “stringent assay conditions” as used herein refers to conditions that are compatible to produce binding pairs of probes and targets of sufficient complementarity to provide for the desired level of specificity in the assay while being incompatible to the formation of binding pairs between binding members of insufficient complementary to provide for the desired specificity. An example of stringent assay conditions is rotating hybridization at 65° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

It will be appreciated that the binding sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater complementarity or identity.

To determine “percent complementarity” or “percent identity” of two nucleic acid sequences, sequences are aligned for optimal comparison purposes (e.g., gaps can be introduced in the sequence of a first nucleic acid sequence for optimal alignment with a second nucleic acid sequence). The nucleotides at corresponding nucleotide positions are then compared. When a position in the first sequence is occupied by a complementary nucleotide as the corresponding position in the second sequence, then the molecules are complementary at that position. Likewise, when a position in the first sequence is occupied by the same nucleotide as the corresponding position in the second sequence, then the molecules are identical at that position. The percent complementarity (or percent identity) between the two sequences is a function of the number of complementary positions (or identical positions) shared by the sequences divided by the total number of positions compared (i.e., % complementarity=number of complementary overlapping positions/total number of positions of the shorter nucleotide times. 100%; and % identity=number of identical overlapping positions/total number of positions of the shorter nucleotide.times.100%).

The determination of percent identity between two sequences can also be accomplished using a mathematical algorithm. A preferred, non-limiting example of a mathematical algorithm utilized for the comparison of two sequences is the algorithm of Karlin and Altschul, Proc. Natl. Acad. Sci. U.S.A. 1990;87:2264-2268, modified as in Karlin and Altschul, Proc. Natl. Acad. Sci. U.S.A. 1993;90:5873-5877. Such an algorithm is incorporated into the NBLAST program of Altschul et al., J. Mol. Biol. 1990;215:403.

In one embodiment, the invention relates to a method for quantitating nucleic acid samples. Methods according to the invention can be used to quantitate unamplified and amplified nucleic acid targets, such as genomic nucleic acids. In one embodiment, the method is used to quantitate products of a whole genome amplification technique or an unbiased or partially biased amplification technique and comprises obtaining a portion of an amplified sample, prior to or after amplification and quantitating one or more sequences in the portion of the sample by a non-enzyme based amplification technique. whole genome amplification technique or an unbiased or partially biased amplification technique

In one embodiment, an enzyme-based amplification technique is used to amplify a complex sample of nucleic acids. Such samples can include greater than about 50 different molecules, greater than about 100 different molecules, greater than about 200 different molecules, greater than about 500 different molecules, greater than about 1000 different molecules, greater than about 5000 different molecules, greater than about 10,000 molecules, greater than about 10⁵ different molecules, or greater than about 10⁶ different molecules, etc. In one aspect, the amplification products of the technique are representative of each of the different molecules, e.g., for a sample of greater than about 50 different molecules there are at least about 50 different sequence amplification products.

Samples can be obtained from a variety of sources. As used herein, a sample includes, but is not limited to, a sample of tissue cell(s) or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components), archival samples, fixed samples (e.g., formalin-fixed samples), paraffin-embedded samples, frozen samples, primary tissue samples, cultured samples, from embryos (e.g., preimplantation embryos), from amniotic fluid, from chorionic villus tissue, from sperm or oocytes, from laser capture microdisection, biopsies, flow cytometry separations, and the like. In one aspect, the nucleic acid sample is from a mammalian source. In another aspect, the nucleic acid sample is from a human.

In one aspect, the nucleic acids comprise genomic nucleic acids such as genomic DNA. A sample of nucleic acids may be prepared using any convenient protocol. In many embodiments, a genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source can comprise genomic DNA representing the entire genome from a particular organism, tissue or cell type or mixture of cell types, developmental stage, and the like.

A given initial genomic source may be prepared from a subject, for example a plant or an animal that is suspected of being homozygous or heterozygous for a deletion or amplification of a genomic region.

In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 Mb, such that the may be about 1 Mb or smaller, e.g., less than about 500 Kb, etc.

Where desired, the initial genomic source may be fragmented, to produced a fragmented genomic source, where the molecules have a desired average size range, e.g., up to about 10 Kb, such as up to about 5 Kb or up to about 1 Kb, where fragmentation may be achieved using any convenient protocol, including but not limited to: mechanical protocols, e.g., sonication, shearing, etc., chemical protocols, e.g., enzyme digestion, etc.

Amplification may or may not occur prior to any fragmentation step.

In one embodiment, an enzyme-based amplification step is used which does not substantially reduce the complexity of the initial genomic source of nucleic acids, e.g., genomic DNA is obtained without a pre-selection step and amplification employs a random set of primers or primers whose complements occur at a desired frequency throughout the genome or whose complements are engineered to be included in a plurality (e.g., all) genomic fragments obtained from a sample (e.g., such as linkers ligated to the ends of genomic fragments). In one aspect, amplification results in an amplified version of virtually the whole genome, if not the whole genome, where the fragmentation, if employed, may be performed pre- or post-amplification.

In certain embodiments, non-reduced complexity nucleic acids are ones in which substantially all, if not all, of the sequences found in the initial genomic source (and organism genome from which the initial source is obtained) are present in the nucleic acid population. By “substantially all” is meant typically at least about 75%, such as at least about 80%, at least about 85%, at least about 90% or more, including at least about 95%, at least about 95% etc, of the total genomic sequences are present in the population, where the above percentage values are number of bases in the population as compared to the total number of bases in the genomic source. Because substantially all, if not all, of the sequences found in the genomic source are present in the sample population of nucleic acids (which can be an amplified population of nucleic acids), the resultant population is not one that is reduced in complexity with respect to the initial genomic template.

Methods for amplifying nucleic sequences using enzymes can vary. In one aspect, genomic nucleic acid is amplified using an isothermal amplification technique. In another aspect, nucleic acid is amplified using a strand displacement technique, such as multiple strand displacement. In a further aspect, the nucleic acid is amplified using random primers, degenerate primers and/or primers which bind to a constant sequence ligated to ends of genomic fragments in a sample.

In one embodiment, a primer set is used that results in the production of a nucleic acid collection of high complexity, i.e., comparable or substantially similar complexity to the initial source nucleic acids (e.g., such as a genomic source). In many embodiments, the above described population of probe nucleic acids in which substantially all, if not all, of the sequences found in the initial genomic source are present, is produced using a primer mixture of random primers, i.e., primers of random sequence. The primers employed in the subject methods may vary in length, and in many embodiments range in length from about 3 to about 25 nucleotides (“nt”), sometimes from about 5 to about 20 nt and sometimes from about 5 to about 10 nt. In certain aspects, “random primers” include a collection of individual oligonucleotides of different sequences, for instance, which can be indicated by the generic formula 5′-XXXXX-3′, wherein X represents a nucleotide residue that was added to the oligonucleotide from a mixture of a definable percentage of each dNTP. For instance, if the mixture contained 25% each of dATP, dCTP, dGTP, and dTTP, the indicated oligonucleotide would contain a mixture of oligonucleotides that have a roughly 25% average chance of having A, C, G, or T at each position. In certain aspects, random primer sequences are random if the conditions of their use cause the locations of their binding to a template nucleic acid to be indeterminate. Also, random primers may be “random” only over a portion of their length. For example, the primers may have constant regions shared by all primers.

The total number of random primers of different sequence that is present in a given population of random primers may vary, and depends on the length of the primers in the set. As such, in the sets of random primers, which include all possible variations, the total number of primers n in the set of primers that is employed is 4^{Y}, where Y is the length of the primers. Thus, where the primer set is made up of 3-mers, Y=3 and the total number n of random primers in the set is 4³ or 64. Likewise, where the primer set is made up of 8-mers, Y=8 and the total number n of random primers in the set is 4⁸ or 65,536. Typically, an excess of random primers is employed, such that in a given primer set employed in the subject invention, multiple copies of each different random primer sequence is present, and the total number of primer molecules in the set far exceeds the total number of distinct primer sequences, where the total number may range from about 1.0×10¹⁰ to about 1.0×10²⁰, such as from about 1.0×10¹³ to about 1.0×10¹⁷, e.g., 3.7×10¹⁵. The primers may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al. (1981), Tetrahedron Letters 22, 1859. One method for synthesizing oligonucleotides on a modified solid support is described in U.S. Pat. No. 4,458,066. Methods for producing random primers are also described in U.S. Pat. Nos. 5,043,272 and 5,106,727, for example. Randomness in a primer sequence may be introduced by providing a mixture of nucleic acid residues in the reaction mixture at one or more addition steps (to produce a mixture of oligonucleotides with random sequence at that residue position). Thus, an oligonucleotide that is random throughout its length can be generated by sequentially incorporating nucleic acid residues from a mixture of 25% of each of dATP, dCTP, dGTP, and dTTP, to form an oligonucleotide. Other ratios of dNTPs can be used (e.g., more or less of any one dNTP, with the other proportions adapted so the whole amount is 100%).

In one embodiment, multiple displacement amplification (MDA) is used to amplify a genomic sample. In one aspect, the method comprises obtaining a genomic nucleic acid sample and contacting the sample with a phi29-like polymerase using random primers or primers complementary to frequently represented sequences in the nucleic acid sample. In one aspect, polymerase comprises a 3-5′ exonuclease proofreading activity. In another aspect, the polymerase comprises an error rate which is less than the error rate of Taq polymerase, e.g., an error rate of less than about 1×10⁻⁴, less than about 1×10⁻⁵, less than about 5×10⁻⁶ (in mutations/nucleotide) in the amplified DNA, or less than about 1×10^{-6.}

In one embodiment, the amount of input genomic DNA is small, comprising less than about 500 ng, less than about 250 ng, less than about 100 ng, less than about 50 ng, less than about 10 ng, less than about 5 ng, or about 1 ng of genomic DNA. In one aspect, the enzyme-based amplification method provides an at least about 1,000-fold amplification of DNA, an at least about 2000-fold amplification, an at least about 5000-fold amplification or an at least about 10,000-fold amplification. In another aspect, genomic DNA is amplified without prior processing steps other than cell lysis and, optionally, dilution, e.g., without centrifugation, addition of chaotropic agents, solvents, alcohols, contacting to a column to remove contaminants and DNA-drying procedures. However, in other aspects, a genomic sample may be contacted to a matrix comprising one or more types of binding molecules for removing undesired sample components. In certain aspects, e.g., where the nucleic acid sample is from an archival source, such as a paraffin-embedded sample or frozen tissue sample, the sample is processed to obtain suitable amounts of template for a subsequent amplification procedure. See, e.g., Loo, et al., Cancer Research 2004;64:8541-8549; Wang, et al., Genome Research 2004;14:2357-2366; Paris, et al., American Journal of Pathology 2003;162(3):763-770.

In certain embodiments, samples are processed to reduce the complexity of the sample. In one aspect, a sample comprises substantially a single chromosome, e.g., such as obtained after flow sorting nucleic acids in a sample source. In other embodiments, nucleic acids are sorted to obtain specific categories of nucleic acids, e.g., such as nucleic acids which bind to one or more nucleic acid binding sequences, such samples may comprise genomic DNA, transcribed molecules (e.g., RNA) or copies thereof. In other aspects, modified DNA sequences are selected (e.g., such as methylated sequences).

In certain aspects, the amplified DNA is added directly to subsequent genetic assays without the need for DNA purification procedures. For example, the amplified DNA can be applied directly to an array as described further below. In one aspect, the amplified DNA is labeled prior to amplification to the array. In certain aspects, a portion of the amplified DNA is removed prior to labeling and before and/or after amplification for evaluation using a non-enzyme based amplification method.

In one embodiment, a sample source (e.g., such as a tissue, cell(s), etc) is lysed in a lysis buffer. In one aspect, the lysis buffer comprises a base such as KOH and the sample is neutralized after lysis in a buffer such as Tris-HCl. A suitable lysis solution includes 400 mM KOH, 10 mM EDTA at pH 8.0, and 50 mM Dithiothreitol. A suitable neutralization solution comprises 800 mM Tris-HCl. In another aspect, a commercial kit for extracting genomic DNA may be used, such as the DNeasy Tissue Kit (QIAGEN) as described in Hosano, et al., 2003, supra.

Nucleic acids may be diluted as necessary. In one aspect, a sample comprises from about 10 μg to about 10 ng in 100 μl of an appropriate reaction buffer, e.g., comprising 37 mM Tris-HCl (pH 7.5); 50 mM KCl; 10 mM MgCl₂; 5 mM (NH4)₂SO₄; 1 mM dATP, dTTP, dCTP, and dGTP; 50 μM exonuclease-resistant primer; 1 unit/mL yeast pyrophosphatase; and 800 units/mL φ29 DNA polymerase, such as described in Hosano, et al., 2003, supra. The nucleic acids are contacted with primers and polymerase under suitable binding conditions to promote binding between the primers and genomic sequences. In one aspect, reactions are incubated at 30° C. for 16 hours and terminated by heating to 65° C. for 3 minutes. Primers can be synthesized as is known in the art using standard β-cyanoethyl phosphoramidite coupling chemistry (see, e.g., Beaucage, et al., Current Protocols in Nucleic Acid Chemistry. John Wiley, New York, N.Y. 2001) and modified as described in Hosano, et al., 2003, supra, or obtained from commercial sources. φ29 DNA polymerase can be obtained from Amersham Biosciences (ABC) or obtained from a recombinant source such as described in U.S. Pat. No. 5,198,543 for example.

In one embodiment, the enzyme used in the enzyme-based amplification procedure includes a helicase. In one aspect, input DNA from a sample (e.g., genomic DNA or DNA copies of RNA molecules) is contacted with a helicase (e.g., such as E. coli UvrD helicase) for unwinding the input DNA. In certain aspects, helicase is used in conjunction with mutL. Unwound DNA is contacted with single-stranded DNA binding proteins (e.g., such as T4 gene 32 protein (available from Roche Applied Science), and the like), and appropriate concentrations of primers (such as random primers, degenerate sequence primers and the like), and contacted with a DNA polymerase (such as an exonuclease-deficient Klenow fragment of DNA polymerase I) in the presence of dNTPs. Samples are incubated under suitable amplification conditions. In one aspect, isothermal conditions such as incubation at 37° C. are employed throughout the procedure. However, in certain aspects, template is heated at 95° C. to denature and brought to 37° C. in 1-4 minutes, for subsequent amplification for approximately 1-2 hours.

Other whole genome amplification methods may be performed such as DOP (Telenius, Genomics 1992;13:718-725) and PEP (Zhang, et al., Proc. Natl. Acad. Sci. 1992; 89:5847-5851), or inter-Alu PCR. In one aspect, an amplification method is selected which provides a minimal amount of bias.

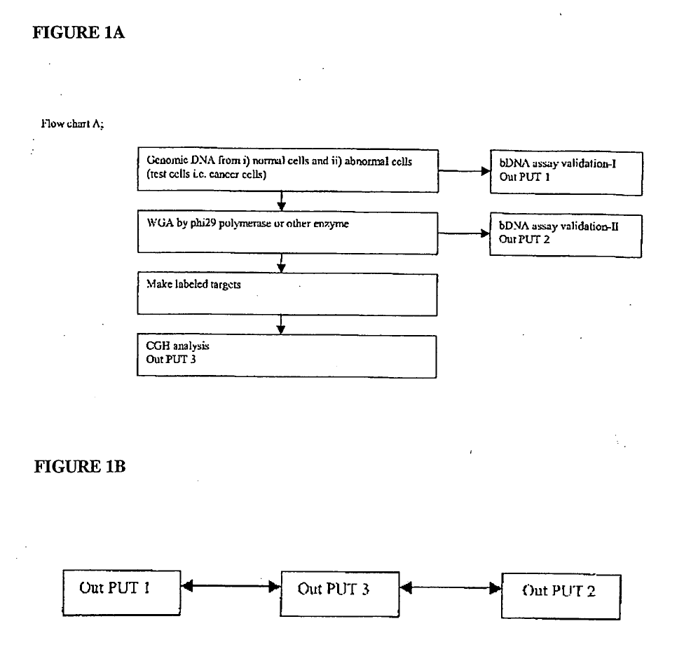

In one embodiment, as illustrated in FIG. 1A, the amount of bias or lack thereof in the amplification method is monitored by performing a non-enzyme based amplification method (e.g., such as a signal amplification method) to amplify selected test sequences. In one aspect, lack of bias is demonstrated where each of a plurality of selected probe sequences in a probe set specifically binds to appropriate target sequences in a portion of the amplified test sample. In another aspect, lack of bias is demonstrated where the relative proportions of bound probes in the probe set corresponds to the relative copy numbers of the target sequences in the test genomic sample. In a further aspect, a lack of bias is demonstrated when the relative proportions of bound probes in the probe set correspond to the relative proportions of bound probes in a probe set contacted to a portion of test genomic sequences prior to their amplification.

In one embodiment, the probe sequences of the probe set have target complements spaced (uniformly or non-uniformly) throughout the genome. In one aspect, a probe set comprises probe sequences representing 47 different loci, one on each p and q arm of the 23 human chromosomes plus one locus on the Y-chromosome. In another aspect, the probe set comprises probe sequences which include repetitive sequences (e.g., such as Alu sequences, centromeric sequences, telomere sequences, LINE sequences, SINE sequences and the like).

In one aspect, the non-enzyme based amplification method is one that relies on signal amplification, e.g., the method comprises contacting nucleic acids in the portion of the sample with a probe set comprising multi-labeled probe sequences. The probe sequences can be labeled directly or indirectly with multiple labels (e.g., using multimeric nucleic acids such as branched DNA molecules).

In one embodiment, the method comprises contacting a first portion of an amplified test nucleic acid sample to an array of probe sequences under specific binding conditions and identifying a first complex formed between a test nucleic acid and a probe sequence on the array. The amount of first complex formed is compared to an amount of a second complex formed between an amplified reference sequence from a reference sample and the same probe sequence and/or the amount of a second complex formed between an amplified reference sequence present at a known level in the amplified test sample (e.g., such as reflecting an amplified diploid amount of the reference sequence) and a different probe sequence. The amount of the first and second complex are compared. For example, the ratio of the amount of the first complex to the second complex can be determined.

A second portion of the amplified test genomic nucleic acid sample is contacted under specific binding conditions to a probe set comprising a multi-labeled probe nucleic acid molecule comprising the probe sequence, wherein the multi-labeled probe nucleic acid molecule comprises a plurality of labels. The amount of a third complex, i.e., that formed between the multi-labeled probe nucleic acid molecule and amplified test nucleic acids in the second sample is determined. In one aspect, the amount of the third complex is compared to the amount of the first and/or second complex. In another aspect, the amount of the third complex is compared to the amount of a fourth complex formed between a reference nucleic acid molecule from a reference sample and the same multi-labeled probe nucleic acid molecule or between a reference nucleic acid molecule present at a known level in the test sample and a different multi-labeled probe nucleic acid molecule. In a further aspect, the ratio of the amount of first complex to the amount of second complex is compared to the ratio of the amount of third complex to the amount of fourth complex.

In another embodiment, a sample of test nucleic acid is obtained prior to its amplification and a portion of the sample is contacted with the multi-labeled probe nucleic acid molecule under specific binding conditions and the amount of a fifth complex formed between the multi-labeled probe nucleic acid molecule and test nucleic acid is determined. The amount of the fifth complex can be compared to amounts of the first, second, third, and/or fourth complex and/or to a sixth complex formed between a portion of an unamplified reference nucleic acid from a reference sample and the same multi-labeled probe or a reference nucleic acid within the portion of the unamplified test nucleic acid sample and a different multi-label probe (e.g., complementary to the reference nucleic acid). See, e.g., as shown in FIGS. 1A and 1B.

In one embodiment, the amplified test nucleic acids are labeled. In one aspect, the amplified test nucleic acids are labeled after removing a portion of the amplified test nucleic acid sample for contacting with the probe set comprising the multi-labeled probe. In another aspect, the amplified test nucleic acids are labeled during amplification but after removing a portion of unamplified sample for contacting with the probe set.

In certain aspects, complexes are distinguished from each other through the use of distinguishable labels, such as spectrally distinguishable labels. In other aspects, complexes are distinguishable from each other by where they are formed, e.g., on one location of a substrate (e.g., an array or support comprising a capture probe) vs. another.

In certain embodiments, e.g., where the amplified reference nucleic acids are from a reference sample, the amplified reference nucleic acids are labeled during or after amplification, with a label which is distinguishable from the label used to label amplified test nucleic acids applied to the array.

In one embodiment, the test sample and reference sample comprises genomic nucleic acids such as genomic DNA. In one embodiment, the test sample and reference sample comprise amplification products produced from a non-biased enzyme-based amplification process, e.g., using primers that bind to sequences present randomly in the nucleic acid molecules of the sample or which are present abundantly (e.g., such as linker sequences added to genomic fragments after a ligation reaction).

In certain aspects, the selection of probe sequences which form the probe set is based upon the formation of first complexes on the array. In one aspect, a probe sequence for the probe set is selected which forms a desired predetermined amount of first complex with an amplified test genomic sample (e.g., such as a labeled amplified test genomic sample). For example, in one aspect, the desired predetermined amount of complex is at least about three standard deviations different from a background amount (such as an amount of test sample which binds to an inter-feature area on the array or to a non-complementary probe). In another aspect, the probe set comprises a plurality of probe sequences. The probe sequences can represent a range of amounts of first complexes observed upon the array, from a background level, to a level significantly above background, to a level approaching saturation for the detection system used to detect the label being used to label test sequences applied to the array. In one aspect, the method comprises identifying a probe sequence on the array for which the ratio of first and second complexes is different from one, and including the probe sequence in the probe set. In another aspect, the method comprises identifying a probe sequence on the array for which the ratio of first and second complexes is one and including the probe sequence in the probe set.

In one embodiment, the nucleic acids in the second portion of the amplified test genomic nucleic acid sample are stably associated with a solid support. In one aspect, the solid support comprises capture oligonucleotides, which hybridize to a portion of the amplified test genomic nucleic acids. In certain aspects, the amplified test genomic nucleic acid samples are ligated to sequences complementary to at least a portion of the capture oligonucleotides or are amplified with primers which include both sequence selective regions for hybridizing to the genomic DNA and constant regions which are designed to be complementary to the capture oligonucleotide sequences and can be used to capture amplified test genomic nucleic acids. Lengths of capture oligonucleotides can vary and in certain aspects can range from at least about 10 bases, at least about 15 bases, at least about 20 bases, or at least about 50 bases. Capture oligonucleotides can be directly or indirectly stably associated with a solid support by means of linker molecules as is known in the art. Solid supports on which the capture oligonucleotides are bound can include any of the type used for arrays. However, in one aspect, the capture oligonucleotides are bound to beads. In certain aspects, the solid support further comprises a means that permits recovery or identification of individual beads or groups of beads. For example, the supports can comprise magnetic or paramagnetic particles and/or can include identifiers (e.g., such as radio tags).

In another embodiment, the capture oligonucleotides are selected to selectively capture only a portion of the amplified test genomic DNA, e.g., that portion comprising sequences complementary to probe sequences in the probe set. Thus, in certain embodiments, a capture oligonucleotide is selected to hybridize to a genomic sequence which is different from the sequence complementary to the probe sequence.

In one aspect, the portion of the test genome to which the capture oligonucleotide molecule binds is at least about 6 bases, at least about 8 bases, at least about 10 bases, or at least about 15 bases. Generally, the portion is sufficiently long to permit stable binding under the assay conditions be used and can vary based on the sequence content of the test genomic DNA.

In certain aspects, test genomic DNA is captured by the capture oligonucleotides through the use of a “capture extender molecule” which comprises a sequence which binds to a portion of the test genomic DNA and a portion of the capture oligonucleotide. In one aspect, the portion of test genome to which the capture extender molecule binds is at least about 6 bases, at least about 8 bases, at least about 10 bases, or at least about 15 bases. Generally, the portion is sufficiently long to permit stable binding under the assay conditions be used and can vary based on the sequence content of the test genomic DNA. Similarly, the portion of the capture extender molecule which binds to the capture oligonucleotide can vary and in one aspect, is at least about at least about 6 bases, at least about 8 bases, at least about 10 bases, or at least about 15 bases. In one aspect, the capture extender molecule comprises a single stranded nucleic acid molecule having a first sequence which is complementary to the test genomic DNA (or a molecule linked thereto) and a second sequence which is complementary to a sequence of the capture oligonucleotide. Generally, the first and second sequences are non-identical and non-complementary under specific binding conditions used. Also, generally, the first and second sequences are non-identical and non-complementary to a sequence to which a probe of the probe set binds.

The solid phase with bound capture probe (and optionally, capture extender):test genomic DNA is optionally separated from unbound materials.

In one aspect, the plurality of label molecules are attached to a probe sequence by hybridizing a multimeric nucleic acid molecule comprising a sequence complementary to a portion of the probe sequence and a repeating site to which a label can be bound to form a multi-labeled probe. The portion of the probe sequence can be unique to the probe sequence or can comprise a sequence common to a plurality (e.g., some or all) of probe sequences of the probe set. The portion of the probe sequence to which the multimeric molecule can vary and in one aspect, is at least about at least about 6 bases, at least about 8 bases, at least about 10 bases, or at least about 15 bases.

In certain aspects, the repeating site comprises a repeating sequence. In one aspect, the repeating sequence comprises a sequence to which a label molecule (such as a nucleic acid probe conjugated to a detectable molecule) can bind directly or indirectly (e.g., through another binding molecule). The multimeric nucleic acid molecule may be composed of DNA, RNA, PNA, LNA, UNA, sequences, modified nucleotides or combinations thereof. In one aspect, the repeating sequences comprise about 10 to 50, or about 15 to 30, nucleotides. In another aspect, the repeating sequences have a GC content of about 20% to about 80%. The number of repeating sequences can vary, e.g., from about 2 to about 1000, from about 10 to about 100, from about 20 to about 50. Repeating sequence units can be covalently linked directly to each other through phosphodiester bonds or through linking agents such as nucleic acid, amino acid, carbohydrate or polyol bridges, or through other cross-linking agents that are capable of cross-linking nucleic acid or modified nucleic acid strands. Alternatively, the multimer may be comprised of repeating sequence units that are not covalently attached, but are bonded in some other manner, e.g., through hybridization. See, e.g., as described in U.S. Pat. No. 5,175,270. The site(s) of linkage may be at the ends of the segment (in either normal, 3′-5′ orientation or randomly oriented) and/or at one or more internal nucleotides in the strand.

Multimeric nucleic acids can be linear or branched. In linear multimeric nucleic acid molecules, repeating units can be linked end-to-end. Branched multimers can take a variety of conformations. For example, two or more or three or more oligonucleotides can branch from a sequence or molecule which is not necessarily a nucleic acid molecule. In certain aspects, branches can be fork-like, or comb-like having a linear backbone with a plurality of oligonucleotide branches extending therefrom. A multimer can comprise a combination of linear portions comprising repeating units and branched portions. In one aspect, the multimer comprises at least two branches, at least three branches, or from at least about 5 to at least about 50 or greater branches. Branches can be single-stranded, double-stranded or partially single stranded. Synthesis and types of multimeric structures are described, for example, in U.S. Pat. No. 5,124,246 and European Patent Publication No. 541,693.

In certain aspects, the probe sequence itself is part of a multimeric sequence. For example, the probe sets can comprise nucleic acid molecules comprising a probe sequence region and repeat sequence regions. In certain aspects, the repeat sequences regions of the probe nucleic acid molecules are constant for each probe while the probe sequence is specific for a particular target molecule. Alternatively, the nucleic acid molecule comprising the probe sequence acts as a label extender binding to a complementary sequence in the nucleic acid molecule comprising a plurality of repeat sequence regions. In certain aspects, the nucleic acid molecule comprising the probe sequence comprises a constant region complementary to a sequence of the nucleic acid molecule comprising the plurality of repeating sequence regions. Accordingly, a single type of nucleic acid multimer may be designed which can bind to a plurality of different sequence nucleic acid molecules comprising probe sequences via the constant region.

In certain aspects, a plurality of repeat sequence units of the multimeric nucleic acid molecules bind directly (e.g., via a covalent linkage) or indirectly (e.g., via a binding partner) to a label molecule. In one aspect, the label molecule comprises a nucleic acid molecule comprising a detectable molecule conjugated thereto. The label molecule can be directly or indirectly detectable. In one aspect, the label molecule comprises a detectable molecule that is an enzyme (e.g., such as alkaline phosphatase). The amount of label can be detected by detecting a reaction catalyzed by the enzyme. As discussed above, the multimeric nucleic acid molecule can comprise the probe sequence or a sequence which hybridizes to a portion of the probe sequence. As used herein, a “multi-labeled nucleic acid molecule comprising a probe sequence” encompasses either possibility.

The solid phase capture probe: target nucleic acid sample is hybridized to a probe set comprising multi-labeled nucleic acid molecules as described above under hybridization conditions permitting the multi-labelled nucleic acid molecules to hybridize specifically to target sequences. In one aspect, the resulting solid phase complex is then separated from unbound molecules by washing. The remaining label is measured.

Complementary sequences having suitable target specificity can be designed using methods and programs known in the art. In one aspect, the Genospectra ProbeDesigner™ Software is used to select and/or design sequences for capture oligonucleotides, capture sequences, probe sequences, label extenders, blocking oligomers and the like

In one embodiment, the probe set comprises a plurality of different multi-labeled nucleic acid molecules comprising different probe sequences. In one aspect, the probe set comprises at least about 5 different probe sequences. In another aspect, the probe set comprises at least about 10 different probe sequences. In one aspect, at least two different probe sequences form different amounts of complexes on the array. In one aspect, the different probe sequences form complexes which differ by an at least one-fold amount.

A variety of labels may be used for generating multi-labeled nucleic acid molecules. Examples of labels include, but are not limited to, fluorescent labels, chemiluminescent labels, and inorganic labels (e.g., gold or ferritin labels) as well as enzymatic labels. Labels may be used that are detectable, for example, by chromogenic detection, chemiluminescent detection or fluorescent detection. Labels that may be used include enzymes such as alkaline phosphatase, β-galactosidase or horseradish peroxidase, which are detected using a chromogenic substrate. For example, alkaline may be detected using 5-bromo-4-chloro-3-indolyl phosphate or nitroblue tetrazolium salt. Other labels include fluorescent tags such as fluorescein, rhodamine, and resorufin, and derivatives thereof, as well as coumarins such as hydroxycoumarin. Additionally fluorescence resonance energy transfer may be measured.

Labels also can be indirectly attached to a nucleic acid molecule via a binding partner. For example, the nucleic acid molecule can be conjugated to biotin and the label can be conjugated to avidin or streptavidin or visa versa. Reagents for labeling streptavidin or avidin with a fluorescent tag include, but are not limited to 5(6)-Carboxyfluorescein-N-hydroxysuccinimide ester (FLUOS), 7-amino-4-methyl-coumarin-3-acetic acid-N′-hydroxysuccinimide ester (AMCA, activated) and fluorescein isothiocyanate (FITC) (available from Boehringer Mannheim, Indianapolis, Ind.). Methods for fluorescently labeling proteins with fluorescent labels, and methods for detection of the fluorescent labels, are known in the art. Other labeled molecules include, but are not limited to, streptavidin-gold, streptavidin-fluorochrome, streptavidin-AMCA, streptavidin-fluorescein, streptavidin-phycoerythrin (STPE), streptavidin-sulforhodamine 101, avidin-FITC and avidin-Texas red® (commercially available from Boehringer Mannheim, Indianapolis, Ind.).

Detection methods used to detect and/or quantitate multi-label nucleic acid probe:sample nucleic acid complexes will vary depending on the nature of the label selected. In one aspect, the presence and/or amount of bound label is detected using a luminometer. In another aspect, the luminometer comprises a heater for maintaining a constant temperature between about 37.0° and 53.0° C.

In certain aspects, the probe set comprises a plurality of multi-labeled nucleic acid molecules, each member of the plurality comprising a different probe sequence. Binding of the different members to the sample nucleic acids can be detected and distinguished by providing different distinguishable labels on each of the different members. Alternatively, or additionally, a portion of a sample to be contacted with the members of the probe set can be divided into a number of portions corresponding to the number of members, for contacting each of the divided portions with a different member of the plurality. In one aspect, the portions are added to different containers or wells of the same container comprising capture probes and the different members of the probe set are added to the different wells. In some aspects, the different members of the probe set are provided in a multi-well container which can be interfaced with a multi-well container comprising the capture probes, e.g., via fluidic connections.

Various techniques can be used to decrease non-specific binding to sequences other than the complements of probes sequences (and/or capture oligonucleotides, multimeric nucleic acids, etc). In one aspect, nucleotide bases are modified to reduce nonspecific binding as described, for example, in U.S. Pat. No. 6,232,462 and Collins, et al., Nucleic Acids Research 1997;25(15):2979-2984. In another aspect, target nucleic acids are additionally contacted with blocking oligomers designed to be complementary to sequences other than sequences complementary to the probe molecules or to the capture oligonucleotides to minimize binding between probe sequences and sequences other than their complements.

In certain embodiments, multi-labeled nucleic acid probes are used to quantitate and/or validate a nucleic acid sample being applied to a nucleic acid array. In one aspect, the relative amount of a first complex formed between a test nucleic acid target and probe and a second complex formed between a reference nucleic acid and the same probe sequence (on the same or a different array) is determined and compared to the relative amount of a multi-label probe:test nucleic acid complex (third complex) and a multi-label probe:reference nucleic acid complex (fourth complex).

In one embodiment, the ratio of first to second complexes on an array is used to determine the copy number of a nucleic acid in a sample (such as a genomic DNA sample). Copy number determination can include the determination of both gain and loss of sequences. In a one aspect, copy number determination is correlated with one or more characteristics of a patient supplying the sample, e.g., such as a disease afflicting the patient or the risk for a patient of developing disease symptoms. In another aspect, copy number determination is associated with changes in characteristics of a patient, e.g., by comparing samples of nucleic acids from a patient obtained at different time periods. Copy number determinations can be used for prenatal testing of chromosomal imbalances (e.g., such as trisomy 21) and both germline and somatic cells can be sample sources for methods of the invention.

In still another aspect, copy number determination is used to screen samples to identify patients at risk for a disease associated with a genomic imbalance such as cancer. In still another aspect, copy number determination is used to screen samples to determine stage and prognosis of a disease associated with a chromosomal imbalance such as cancer. Additional applications are further described in U.S. Pat. Nos. 6,335,167; 6,197,501; 5,830,645; and 5,665,549; the disclosures of which are herein incorporated by reference.

In certain aspects, the array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. In certain aspects, the array comprises both non-coding and coding sequences. In one aspect, individual probes on the array comprise either coding or non-coding sequences but do not comprise both coding and non-coding sequences. In certain aspects, individual probes comprise sequences that do not normally occur together, e.g., to detect gene rearrangements, for example.

As discussed previously, nucleic acid samples can be obtained from a variety of sample sources. In one aspect, a test sample is from a biopsy. In another aspect, the test sample is from a tumor. In still another aspect, the sample is from a source of fetal nucleic acids, such as amniotic fluid or chorionic villus cells. The source of reference nucleic acid also can vary. In one aspect, the reference nucleic acid is from a healthy patient. In another aspect, the reference nucleic acid is from an individual known to have a diploid complement of a nucleic acid or a know amount of the nucleic acid. Thus, in certain aspects, the reference nucleic acid is from the test sample. In one aspect, the reference sequence includes one or more paralogous sequences which correspond to test sequences.

Test and reference nucleic acids applied to an array may or may not be labeled, depending on the particular detection protocol employed in a given assay. For example, in certain embodiments, binding events on the surface of a substrate may be detected by means other than by detection of a labeled probe nucleic acids, such as by change in conformation of a conformationally labeled immobilized probe, detection of electrical signals caused by binding events on the substrate surface, etc. In certain embodiments, however, the populations of target nucleic acids are labeled (e.g., test and reference nucleic acids from test and reference samples sources, respectively), where the populations may be labeled with the same label or different labels, depending on the actual assay protocol employed. For example, where each population is to be contacted with different but identical arrays, each target nucleic acid population or collection may be labeled with the same label. Alternatively, where both populations are to be simultaneously contacted with a single array of probes, i.e., cohybridized to the same array of immobilized probe nucleic acids, the populations are generally distinguishably or differentially labeled with respect to each other. In some aspects, there may be more than one test nucleic acid sample and more than one reference nucleic acid sample.

In one aspect, two or more (i.e., at least first and second, where the number of different collections may, in certain embodiments, be three, four or more) populations of target nucleic acids are prepared from different genomic sources and a collection of labeled target nucleic acids are prepared for application to one or more arrays.

A genomic source may be prepared using any convenient protocol. In many embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in many embodiments of interest, genomic DNA representing the entire genome from a particular organism, tissue or cell type. (However, in those embodiments where the collection of probe nucleic acids is one of reduced complexity with respect to the genome of the organism from which it is prepared, as described in greater detail below, it may comprise a portion of the genome).

A given initial genomic source may be prepared from a subject, for example a plant or an animal, that is suspected of being homozygous or heterozygous for a deletion or amplification of a genomic region. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 MB, such that the may be about 1 Mb or smaller, e.g., less than about 500 Kb, etc. Where desired, the initial genomic source may be fragmented as desired, to produce a fragmented genomic source, where the molecules have a desired average size range, e.g., up to about 10 Kb, such as up to about 1 Kb, where fragmentation may be achieved using any convenient protocol, including but not limited to: mechanical protocols, e.g., sonication, shearing, etc., chemical protocols, e.g., enzyme digestion, etc.

As discussed above, the initial genomic source may be amplified by an enzyme-based amplification procedure, where the amplification may or may not occur prior to any fragmentation step. In those embodiments where the prepared nucleic acid has substantially the same complexity as the initial genomic source from which it is prepared, the amplification step employed is one that does not reduce the complexity, e.g., one that employs a set of random primers, degenerate primers, ligated primers comprising constant regions, and the like. For example, the initial genomic source may first be amplified in a manner that results in an amplified version of virtually the whole genome, if not the whole genome, before labeling, where the fragmentation, if employed, may be performed pre- or post-amplification.

As discussed above, in one aspect, the prepared collection of nucleic acids to be applied to an array is a “non-reduced-complexity” collection of nucleic acids, as compared to the initial genomic source and genome of the organism from which the initial genomic source is obtained. A non-reduced complexity collection is one that is not produced in a manner designed to reduce the complexity of the sample, e.g., is not produced using collections of primers that are designed to prime only a certain percentage or fraction of the initial genomic source. In contrast, a reduced complexity collection of nucleic acids is one that has been produced by a protocol that only amplifies a certain portion, fraction or region of the genomic source used to prepare the collection or is selected (e.g., by a sorting procedure) to remove certain classes of nucleic acids from a sample (e.g., such as nucleic acids which do not bind to a binding protein, as discussed further below or flow-sorted chromosomes, and the like).

In certain embodiments, non-reduced complexity collections of nucleic acids are ones in which substantially all, if not all, of the sequences found in the initial genomic source (and organism genome from which the initial source is obtained) are present in the prepared population of nucleic acids being applied to the array. By substantially all is meant typically at least about 75%, such as at least about 80%, at least about 85%, at least about 90% or more, including at least about 95%, at least about 95% etc, of the total genomic sequences are present in the prepared population to be applied to the array, where the above percentage values are number of bases in the prepared population as compared to the total number of bases in the genomic source. Because substantially all, if not all, of the sequences found in the genomic source are present in the prepared population of nucleic acids, the resultant population of nucleic acids is not one that is reduced in complexity with respect to the initial genomic template, i.e., it is not a reduced complexity population of nucleic acids.

A non-reduced complexity collection of prepared nucleic acids can be identified or validated by screening the collection using a genome wide array of probe nucleic acids for the genomic source of interest. Thus, one can tell whether a given collection of nucleic acids has non-reduced complexity with respect to its genomic source by assaying the collection with a genome wide array for the genomic source. The genome wide array of the genomic source is an array of probe nucleic acids in which the entire genomic source is screened at a sufficiently high resolution, where the resolution is typically at least about 1 Mb, e.g., at least about 500 Kb, such as at least about 250 Kb, including at least about 100 Kb, e.g., 50 Kb or higher (such as 25 Kb, 15 Kb, 10 Kb or higher), where resolution in this context means lengths of the genomic source between regions present on the array in the form of immobilized probes. In such a genomic wide assay of sample, a non-reduced complexity sample is one in which substantially all of the array features on the array provide a positive signal, where by substantially all is meant at least about 50%, such as at least about 60, 70, 75, 80, 85, 90 or 95% (by number) or more.

However, in an additional or alternative embodiment, the non-reduced complexity of the prepared nucleic acids is identified or validated by a non-enzyme based amplification method using a probe set as discussed above to selectively detect and quantitate the presence of representative test sequences in the sample.

In certain embodiments, as discussed above, the prepared population of nucleic acids being applied to the array is labeled with a detectable label. In one aspect, the labeling procedure does not alter the complexity of the sample to any significant extent as compared to the initial unlabeled source (e.g., such as the initial genomic source). A number of different nucleic acid labeling protocols are known in the art and may be employed to produce a population of labeled probe nucleic acids. The particular protocol may include the use of labeled primers, labeled nucleotides, modified nucleotides that can be conjugated with different dyes, a non-amplifying primer extension protocol (e.g., a single product is produced per template strand), one or more amplification steps, etc. In certain aspects, it is desirable to remove the portion of the sample from the sample for evaluation by the non-enzyme-based amplification step prior to the labeling step, which incorporates label for detecting binding to the array. However, in other aspects, the portion is removed after labeling and the types of labels used in the multi-labeled probe sets are spectrally distinguishable from those used for detecting binding to the array.

In one aspect, in generating labeled nucleic acids for application to an array, the nucleic acid template (e.g., genomic DNA) and random primer population are employed together in a primer extension reaction that produces labeled nucleic acids. Primer extension reactions for generating labeled nucleic acids are well known to those of skill in the art, and any convenient protocol may be employed. Primers are contacted with the genomic template in the presence of a sufficient DNA polymerase under primer extension conditions sufficient to produce the desired primer extension molecules. DNA polymerases of interest include, but are not limited to, polymerases derived from E. coli, thermophilic bacteria, archaebacteria, phage, yeasts, Neurosporas, nemotodes, Drosophila sp, primates and rodents, as well as polymerases derived synthetically from several species or by in silico modeling. The DNA polymerase extends the primer according to the genomic template to which it is hybridized in the presence of additional reagents which may include, but are not limited to: dNTPs; monovalent and divalent cations (e.g. KCl, MgCl₂); sulfhydryl reagents (e.g. dithiothreitol); and buffering agents, e.g. Tris-HCl.

In one aspect, the reagents employed in the subject primer extension reactions include a labeling reagent, where the labeling reagent may be the primer or a labeled nucleotide, which may be labeled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g., dCTP. Fluorescent moieties which may be used to tag nucleotides for producing labeled probe nucleic acids include, but are not limited to: fluorescein, the cyanine dyes, such as Cy3, Cy5, Alexa 555, Bodipy 630/650, and the like. Other labels may also be employed as are known in the art.

In one aspect, the genomic template is subjected to a strand disassociation condition, e.g., subjected to a temperature ranging from about 80° C. to about 100° C., usually from about 90° C. to about 95° C. for a period of time, and the resultant disassociated template molecules are then contacted with the primer molecules under annealing conditions, where the temperature of the template and primer composition is reduced to an annealing temperature of from about 20° C. to about 80° C., usually from about 37° C. to about 65° C. In certain embodiments, a “snap-cooling” protocol is employed, where the temperature is reduced to the annealing temperature, or to about 4° C. or below in a period of from about 1 second to about 30 seconds, usually from about 5 seconds to about 10 seconds.

The resultant annealed primer/template hybrids are then maintained in a reaction mixture that includes the above-discussed reagents at a sufficient temperature and for a sufficient period of time to produce the desired labeled probe nucleic acids. Typically, this incubation temperature ranges from about 20° C. to about 75° C., usually from about 37° C. to about 65° C. The incubation time typically ranges from about 5 min to about 18 hours, usually from about 1 hour to about 12 hours.

In one embodiment, at least a first collection of target nucleic acids and a second collection of target nucleic acids are produced from two different genomic sources, e.g., a reference and test genomic template. As indicated above, depending on the particular assay protocol (e.g., whether both populations are to be hybridized simultaneously to a single array or whether each population is to be hybridized to two different but substantially identical, if not identical, arrays) the populations may be labeled with the same or different labels. As such, a feature of certain embodiments is that the different collections or populations of produced labeled probe nucleic acids are all labeled with the same label, such that they are not distinguishably labeled. In yet other embodiments, a feature of the different collections or populations of produced labeled probe nucleic acids is that the first and second labels are distinguishable from each other. The constituent probe members of the above produced collections can range in length from about 100 to about 10,000 nt, such as from about 200 to about 10,000 nt, including from about 100 to 1,000 nt, from about 100 to about 500, etc.

The labeled nucleic acid collections (e.g., test nucleic acids sample comprising internal reference nucleic acids or test and reference nucleic acid samples of substantially equal complexity) are contacted (separately or to a plurality of arrayed probes under conditions such that nucleic acid hybridization to the probes can occur. The collections can be contacted to the probes either simultaneously or serially. Depending on how the collections are labeled, the collections may be contacted with the same array or different arrays. In one aspect, where when the collections or populations are contacted with different arrays, the different arrays are substantially, if not completely, identical to each other in terms of feature content and, in certain aspects, organization.

In one embodiment, the arrayed probes comprise oligonucleotides. By oligonucleotide is meant a nucleic acid having a length ranging from about 10 to about 200 including from about 10 or about 20 to about 150 nt, where in many embodiments the target nucleic acids range in length from about 50 to about 90 nt or about 50 to about 80 nt, such as from about 50 to about 70 nt.

Probe nucleic acids on the array can be derived from virtually any source. In one aspect, the probes comprise nucleic acid molecules having sequences derived from representative locations along a chromosome of interest, an entire genome of interest, a cDNA library, and the like.

The choice of nucleic acids to use as probes may be influenced by prior knowledge of the association of a particular chromosome or chromosomal region with certain disease conditions. International Application WO 93/18186 provides a list of exemplary chromosomal abnormalities and associated diseases, which are described in the scientific literature. Alternatively, whole genome screening to identify new regions subject to frequent changes in copy number can be performed using the methods of the present invention. Accordingly, probes on the array can contain nucleic acids representative of locations distributed over the entire genome. In such embodiments, the resolution may vary from at least about 500 Kb, such as at least about 250 Kb, at least about 200 Kb, at least about 150 Kb, at least about 100 Kb, at least about 50 Kb, including at least about 25 Kb, at least about 10 Kb or higher. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for prenatal testing. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome).

Other “themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning.

Of interest, in constructing the arrays, are both coding and non-coding genomic regions, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the targets directed to non-coding regions and others directed to coding regions. In certain embodiments, one can have all of the targets directed to non-coding sequences. In certain embodiments, one can have all of the targets directed to coding sequences.

Oligonucleotide probes can be arrayed on a variety of substrates, as discussed above, including, but not limited to, a membrane, glass, plastic, capillary, microfluidic channel, web, pins, or a bead. Probes may be covalently bound or noncovalently attached through nonspecific binding, adsorption, physisorption or chemisorption. The immobilization of nucleic acids on solid support surfaces is discussed more fully below.

A wide variety of organic and inorganic polymers, as well as other materials, both natural and synthetic, may be employed as the material for the solid surface. Illustrative solid surfaces include nitrocellulose, nylon, glass, fused silica, diazotized membranes (paper or nylon), silicones, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition substances that form gels can be used. Such materials include proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, enhance signal detection or the like.

If covalent bonding between a compound and the surface is desired, the surface will usually include appropriate functionalities to provide for the covalent attachment. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. For example, methods for immobilizing nucleic acids by introduction of various functional groups to the molecules is known (see, e.g., Bischoff et al., Anal. Biochem. 1987;164:336-344; Kremsky et al., Nuc. Acids Res. 1987;15:2891-2910). Modified nucleotides can be placed on the target using PCR primers containing the modified nucleotide, or by enzymatic end labeling with modified nucleotides, or by non-enzymatic synthetic methods.

In one embodiment, membrane supports (e.g., nitrocellulose, nylon, polypropylene) for the nucleic acid arrays of the invention are used. Such membranes are generally available and protocols and equipment for hybridization to membranes is well known. Many membrane materials, however, have considerable fluorescence emission, where fluorescent labels are used to detect hybridization. To optimize a given assay format one of skill can determine sensitivity of fluorescence detection for different combinations of membrane type, fluorochrome, excitation and emission bands, spot size and the like. In addition, low fluorescence background membranes have been described (see, e.g., Chu et al., Electrophoresis 1992;13:105-114).

The sensitivity for detection of spots of various diameters on such membranes can be readily determined by, for example, spotting a dilution series of fluorescently end-labeled DNA fragments. These spots are then imaged using conventional fluorescence microscopy. The sensitivity, linearity, and dynamic range achievable from the various combinations of fluorochrome and membranes can thus be determined. Serial dilutions of pairs of fluorochrome in known relative proportions can also be analyzed to determine the accuracy with which fluorescence ratio measurements reflect actual fluorochrome ratios over the dynamic range permitted by the detectors and membrane fluorescence.

Arrays on substrates with much lower fluorescence than membranes, such as glass, quartz, or small beads, can achieve much better sensitivity. For example, elements of various sizes, ranging from about 1 mm diameter down to about 1 μm can be used with these materials. Small array members containing small amounts of concentrated probes are conveniently used for high complexity comparative hybridizations since the total amount of target available for binding to each element will be limited. Thus it may be advantageous in certain embodiments to have array features that contain a small amount of concentrated probe so that the signal that is obtained is highly localized and bright. Such small features are typically used in arrays with densities greater than 10⁴/cm². Relatively simple approaches capable of quantitative fluorescent imaging of 1 cm² areas have been described that permit acquisition of data from a large number of members in a single image (see, e.g., Wittrup et al., Cytometry 1994;16:206-213).

Covalent attachment of probes to glass or synthetic fused silica can be accomplished according to a number of known techniques. Such substrates provide a very low fluorescence substrate, and a highly efficient hybridization environment.

There are many possible approaches to coupling nucleic acids to glass that employ commercially available reagents. For instance, materials for preparation of silanized glass with a number of functional groups are commercially available or can be prepared using standard techniques. Alternatively, quartz cover slips, which have at least 10-fold lower auto fluorescence than glass, can be silanized. In certain embodiments of interest, silanization of the surface is accomplished using the protocols described in U.S. Pat. No. 6,444,268, the disclosure of which is herein incorporated by reference, where the resultant surfaces have low surface energy that results from the use of a mixture of passive and functionalized silanization moieties to modify the glass surface. Additional linking protocols of interest include, but are not limited to: polylysine as well as those disclosed in U.S. Pat. No. 6,319,674, the disclosure of which is herein incorporated by reference. The probes can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using, e.g., protein A, following standard protocols (see, e.g., Smith et al., Science 1992;258:1122-1126). Biotin or digoxigenin end-labeled nucleic acids can be prepared according to standard techniques. Hybridization to nucleic acids attached to beads is accomplished by suspending them in the hybridization mix, and then depositing them on the glass substrate for analysis after washing. Alternatively, paramagnetic particles, such as ferric oxide particles, with or without avidin coating, can be used.

In one embodiment, the copy number of particular nucleic acid sequences in two sample collections (e.g., test and reference samples) are compared by hybridizing the nucleic acids to one or more nucleic acid arrays, as described above. In one aspect, the hybridization signal intensity, and the ratio of intensities, produced by the formation of probe:target complexes is determined. Since signal intensities on a target element can be influenced by factors other than the copy number of a nucleic acid sequence in solution, for certain embodiments, an analysis is conducted where two labeled populations are present with distinct labels. Thus, comparison of the signal intensities for a specific probe:target complexes permits a direct comparison of copy number for a given sequence. Different probe:target complexes will reflect the copy numbers for different sequences in the sample populations. The comparison can reveal situations where each sample includes a certain number of copies of a sequence of interest, but the numbers of copies in each sample are different. The comparison can also reveal situations where one sample is devoid of any copies of the sequence of interest, and the other sample includes one or more copies of the sequence of interest. In certain embodiments, the comparison of probe:target complexes formed on the array is done in parallel with the comparison of complexes formed between multi-labeled probes of the probe sets and targets. In other embodiments, the comparison or probe:target complexes formed on the array is done prior to or after comparison of complexes formed between multi-labeled probes of the probe sets and target. In still other embodiments, the identification of probe:target complexes on the array is used in the selection of probes for the probe sets.

In one aspect, standard hybridization techniques (e.g., using high stringency hybridization conditions) are used to probe an array. Suitable methods are described in references describing CGH techniques (Kallioniemi et al., Science 1992;258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol. 1981;21:470-480 and Angerer et al., In Genetic Engineering: Principles and Methods, Setlow and Hollaender, Eds. Vol 7, pgs 43-65 (Plenum Press, New York 1985). See also U.S. Pat. Nos. 6,335,167; 6,197,501; 5,830,645; and 5,665,549; the disclosures of which are herein incorporated by reference.

In one aspect, the array is treated prior to contacting with target nucleic acids to increase the accessibility of target DNA. In another aspect, the array is treated to reduce nonspecific binding. Hybridization may include agitation of the immobilized probes and the target nucleic acids, where the agitation may be accomplished using any convenient protocol, e.g., shaking, rotating, spinning, and the like. Hybridization is allowed to occur under appropriate stringency conditions, e.g., such as high stringency. The term “highly stringent hybridization conditions” as used herein refers to conditions that are compatible to produce complexes between complementary binding members, i.e., between immobilized probes and complementary sample nucleic acids, but which does not result in any substantial complex formation between non-complementary nucleic acids (e.g., any complex formation which cannot be detected by normalizing against background signals to interfeature areas and/or control regions on the array). In one aspect, the contacted array is washed to remove nucleic acid molecules not bound in specific hybridization complexes to the array. Washing may be performed using any convenient washing protocol, where the washing conditions are typically stringent, as described above.

In one aspect, reading of the resultant hybridized array may be accomplished by illuminating the array and reading the location and intensity of signal (e.g., fluorescence) at each feature of the array to detect any binding complexes on the surface of the array. For example, a scanner may be used for this purpose which is similar to the AGILENT MICROARRAY SCANNER available from Agilent Technologies, Palo Alto, Calif. Other suitable devices and methods are described in U.S. patent applications: Ser. No. 09/846,125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of the probe nucleic acids, and are suitable for some embodiments.

Results from the reading or evaluating may be raw results (such as fluorescence intensity readings for each feature in one or more color channels) or may be processed results, such as obtained by subtracting a background measurement, or by rejecting a reading for a feature which is below a predetermined threshold and/or forming conclusions based on the pattern read from the array (such as whether or not a particular target sequence may have been present in the sample, or whether or not a pattern indicates a particular condition of an organism from which the sample came). In certain aspects, a reading may be rejected if representation of a region (or lack of representation in the case of a deletion) in a target nucleic acid sample being scanned by the probe cannot be validated by using one or more selected probes in a multi-labeled probe set. In one aspect, Single copy number differences or change in the amount of a sequence of interest between any two given samples can be detected using arrays as described above.

In certain embodiments, data obtained from methods according to aspects of the invention are transmitted to a remote location. By “remote location” is meant a location other than the location at which the array is present and hybridization occur. For example, a remote location could be another location (e.g. office, lab, etc.) in the same city, another location in a different city, another location in a different state, another location in a different country, etc. Data can be transmitted through signals (e.g., electrical, optical, radio, etc.) over a suitable communication channel (for example, a private or public network).

In other embodiments while amplification of a nucleic acid sample is unbiased, the complexity of the sample is reduced compared to a cell from which the nucleic acid is obtained. For example, in one aspect, prior to amplification, sample nucleic acid sequences (genomic sequences or RNA sequences) are selected by their ability to bind to one or more nucleic acid binding proteins (e.g., proteins which bind to origins of replication, recombination hotspots, promoters, enhancers, untranslated regions, introns or exon/intron boundaries including intronic sequences, methylation sites, and the like). In certain aspects, the sequences which bind to the one or more nucleic acid binding proteins are then amplified using a non-biased amplification method (e.g., such as MDA) and applied to the array. Complexes formed between the amplified sequences and probes on the array are used to identify the types and/or genomic locations of sequences, which are bound to the nucleic acid binding proteins.

In certain aspects, nucleic acid sequences bound to nucleic acid binding proteins are cross-linked to the proteins and cleaved with a cleavage agent (such as an exonuclease) to remove or decrease the amount of nucleic acid sequences outside of the binding region to which the proteins are bound or subjected to shearing conditions as described, for example, in U.S. Pat. No. 6,410,243, the entirety of which is incorporated by reference herein. In certain aspects, proteins in protein complexes are also crosslinked to each other and the complex is cross-linked to a nucleic acid molecule to which it binds. Bound (and optionally crosslinked complexes) can be removed from non-bound nucleic acids and amplified and the amplified nucleic acids corresponding to these binding regions are then applied to the array. In certain aspects, crosslinks are reversed prior to amplification. As above, methods according to the invention can be used to quantify selected sequences in the amplified sample and/or can be used to validate the lack of bias or to determine an amount of bias in the amplification step.

In still other embodiments, complexity of the target is reduced by selecting for particular chromosomes, e.g., by flow sorting.

In further embodiments, complexity of the target is reduced by eliminating non-repetitive sequences, e.g., by denaturing a nucleic acid sample, and separating fractions according to their kinetics of reassociation.

In still further embodiments, reduction in complexity is achieved through the enzyme-based amplification procedure being used, e.g., by contacting sample nucleic acids with primers whose complements are present in the sample at a lower frequency than the complements of primers used to prepare a non-reduced complexity sample.

In one aspect, a reduced complexity sample is one in which the complexity of nucleic acids in the sample is at least about 20-fold less, such as at least about 25-fold less, at least about 50-fold less, at least about 75-fold less, at least about 90-fold less, at least about 95-fold less, than the complexity of the initial source, in terms of total numbers of sequences found in the prepared sample to be applied to the array as compared to the initial source, up to an including a single gene locus being represented in the collection.

In still other embodiments, expressed sequences are obtained from the same sample sources from which genomic sequences are obtained, and changes in expression of genes are evaluated in parallel or sequentially (before or after) with the evaluation of genome-wide changes in copy number or regulatory events (e.g., such as binding of nucleic acid binding proteins to particular regions of the genome). By providing both coding and non-coding sequences on an array, a single array can be used for combined expression/CGH and or location analyses, e.g., by labeling expressed sequence (or copies thereof) with a label, which is distinguishable from a label used to label genomic sequences present in the sample. Multiple, substantially identical arrays can also be used to minimize the numbers of different types of labels. In certain aspects, the expression levels observed through using the array can be validated using multi-labeled probes according to the invention.

Kits

The invention also relates to kits for facilitating the above methods. In one embodiment, the invention provides a kit comprising a probe set for use in a method according to the invention. In one aspect, the kit comprises a plurality of probe sets, each in separate containers. In one aspect, the kit comprises a first probe set and a second probe set, wherein the first probe set comprises probe sequences for scanning a region of the genome at a higher resolution than the second probe set. For example, in one aspect, the second probe set comprises multi-labeled probe sequences which bind to a plurality of chromosomes, whereas the first probe set comprises multi-labeled probe sequences that bind to a fewer than the plurality of chromosomes in the second set, e.g., such as a single chromosome or a single chromosome arm. In one aspect, the probe set comprises sequences whose complements are uniquely present in the X chromosome and/or Y chromosome. In another aspect, the probe set comprises sequences whose complements are uniquely present in at least one, at least two, at least three, at least five, at least 20, or at least each chromosome defining a haploid genome of an organism. In certain aspects, the probe set comprises sequences whose complements are uniquely present in each of distinguishable alleles of a diploid genome.

In one embodiment, the probe sequence of the probe set is part of a multimeric sequence which comprises repeating sequence units in addition to the probe sequence, to which a label can bind directly or indirectly (e.g., through a labeled sequence which bindings to the multimeric sequence). In another embodiment, the probe sequence of the probe set binds to a complementary sequence which is part of a multimeric sequence comprising repeating sequence units. In this case, both the probe sequence of the probe set and the complementary multimeric sequence to which a plurality of labels may be attached can be provided in the same or different containers. In a further embodiment, the probe or probe-specific complement portion of the multimeric nucleic acid molecule can be ligated to a multimeric sequence which can be common to all members of the probe set. Accordingly, the kit may further contain reagents necessary for performing the ligation reaction. Additionally, the kit can comprise reagents suitable for binding the probe sequence to a sample nucleic acid and/or to a multimeric sequence and/or to additional labeling sequences. In still another aspect, the kit comprises capture olignonucleotides (optionally pre-attached to a solid support). In a further aspect, the kit comprises a solid support for attachment to the oligonucleotides.

In another embodiment, the kit comprises reagents for performing a signal-based amplification method and an enzyme-based amplification method.

In still another embodiment, the kit comprises reagents for performing a substantially unbiased enzyme-based amplification method, e.g., such as a whole genome amplification method (e.g., such as MDA). In one aspect, the reagents include a phi29-like enzyme and/or other reagents for performing an isothermal amplification method. In another aspect, the kit comprises random or degenerate primers or primers and linkers suitable for performing ligation-mediated PCR. In still another aspect, the kit comprises a helicase and/or a single-stranded DNA binding protein.

In a further embodiment, the kit comprises reagents for crosslinking nucleic acids to proteins (and optionally, proteins to proteins). In still a further aspect, the kit comprises a binding molecule for sorting bound nucleic acids from non-bound nucleic acids. Suitable binding molecules include, but are not limited to antibodies, antigen-binding fragments thereof, affibodies, aptamers, and the like.

The kit may additionally comprise molecules for performing a primer extension reaction to label amplified nucleic acids or nucleic acids to be labeled. In one aspect, the kit comprises a Cy3 and/or Cy5 dye or another pair of spectrally distinguishable dyes.

Where the kits are specifically designed for use in CGH applications, the kits may further include labeling reagents for making two or more collections of distinguishably labeled nucleic acids according to the subject methods.

In still a further embodiment, the kit comprises an array for use in a method according to aspects of the invention. In one aspect, the array can be used to scan an entire genome. In another aspect, the array can be used to scan subregions of a genome (e.g., particular chromosomes or chromosome arms). In one aspect, probes on the array comprise both coding and non-coding regions.

In a certain aspects, the kit includes a multi-well plate (e.g., such as a microtiter plate) comprising capture oligonucleotides stably associated with a least a portion of a plurality of the wells. The capture probes can be used to immobilize sample nucleic acids to be contacted with a probe set. In another aspect, the multi-well plate comprises at least one array at the base of a well which may be attached to the base of the well or otherwise stably associated with the base of the well. However, in certain aspects, the array merely rests on the base of the well. In still other aspects, the base of several wells comprises a single substrate comprising a plurality of arrays, each array bounded by the walls of a well. In certain aspects, the multi-well plates comprise both arrays and capture probes in different wells. In a further aspect, e.g., where the probe set comprises a plurality of multi-labeled nucleic acid molecules, each member of the plurality comprising a different probe sequence, the different members are provided in different containers or in different wells in a multi-welled container. In certain aspects, the multi-welled container comprising the different members of a probe set can be interfaced with a multi-welled container comprising capture oligonucleotides, e.g., via fluidic connections.

Finally, the kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method comprising:
contacting a first portion of an amplified test nucleic acid sample to an array of probe sequences, identifying a first complex formed between a test nucleic acid and a probe sequence on the array, comparing an amount of the first complex to the amount of a second complex formed between an amplified reference nucleic acid and the same or a different probe sequence;
contacting a second portion of the amplified test nucleic acid sample under specific binding conditions to a probe set comprising a multi-labeled probe nucleic acid molecule comprising the probe sequence, wherein the multi-labeled probe nucleic acid molecule comprises a plurality of labels;
determining an amount of a third complex formed between the multi-labeled probe nucleic acid molecule and test nucleic acids in the second portion; and
comparing the amount of third complex to the amount of the first and/or second complex.

2. The method of claim 1, further comprising comparing the amount of third complex to an amount of a fourth complex formed between a reference nucleic acid and the multi-labeled probe nucleic acid molecule.

3. The method of claim 2, further comprising comparing the ratio between the amount of the first complex to the amount of the second complex to the ratio between the amount of the third complex to the amount of the fourth complex.

4. The method of claim 1, wherein the amount of the third complex is compared to the amount of the first and the second complex.

5. The method of claim 1, wherein a sample of test nucleic acid is obtained prior to its amplification, and a portion of the sample is contacted with the multi-labeled probe nucleic acid molecule under specific binding conditions and the amount of a fifth complex formed between the multi-labeled probe nucleic acid molecule and test genomic nucleic acid is determined.

6. The method of claim 5, wherein the amount of fifth complex is compared to the amount of a sixth complex formed between a reference nucleic acid and the multi-labeled probe nucleic acid molecule.

7. The method of claim 1, wherein the method comprises identifying a probe sequence on the array for which the ratio of first and second complexes is different from one and including the probe sequence in the probe set.

8. The method of claim 1, wherein the method comprises identifying a probe sequence on the array for which the ratio of first and second complexes is one and including the probe sequence in the probe set.

9. The method of claim 7, wherein the method comprises identifying a probe sequence on the array for which the ratio of first and second complexes is one and including the probe sequence in the probe set.

10. The method of claim 1, wherein nucleic acids in the second portion of the amplified test nucleic acids are stably associated with a solid support.

11. The method of claim 10, wherein the solid support comprises capture oligonucleotides which hybridize to a portion of the amplified test nucleic acids.

12. The method of claim 10, wherein the amplified test nucleic acid sequences are ligated to sequences complementary to at least a portion of the capture oligonucleotides.

13. The method of claim 1, wherein the probe set comprises a plurality of multi-labeled probe nucleic acid molecules, each member of the plurality comprising a different probe sequence, and wherein the second portion of the amplified test nucleic acid sample is divided into a number of portions corresponding to the number of members for contacting each of the divided portions with a different member of the plurality.

14. The method of claim 1, wherein the plurality of label molecules are attached to the probe sequence by a branched nucleic acid molecule comprising a sequence complementary to a portion of the probe sequence.

15. The method of claim 14, wherein a plurality of branches of the branched nucleic acid molecule bind to a label molecule.

16. The method of claim 15, wherein the label molecule comprises a nucleic acid molecule comprising a detectable molecule conjugated thereto.

17. The method of claim 16, wherein the detectable molecule is directly detectable.

18. The method of claim 16, wherein the detectable molecule is an enzyme and is detected by detecting a reaction catalyzed by the enzyme.

19. The method of claim 1, wherein the probe set comprises at least about 5 different probe sequences.

20. The method of claim 1, wherein the probe set comprises at least about 10 different probe sequences.

21. The method of claim 1, wherein the probe set sequences comprise a plurality of different probe sequences.

22. The method of claim 1, wherein the plurality of different probe sequences correspond to probes in different first complexes formed on the array.

23. The method of claim 22, wherein the plurality comprises at least about five different probe sequences.

24. The method of claim 22, wherein the plurality comprises at least about ten different sequences.

25. The method of claim 22, wherein the plurality comprises at least two different sequences which form different amounts of first complexes on the array.

26. The method of claim 25, wherein the plurality comprises at least two different sequences forming first complexes which differ by an at least about one-fold amount.

27. The method of claim 25, wherein the plurality comprises a probe sequence forming an amount of first complex that differs by at least about three standard deviations from a background amount.

28. The method of claim 1, wherein the test nucleic acid is amplified using an isothermal amplification technique.

29. The method of claim 1, wherein the test nucleic acid is amplified using a multiple strand displacement technique.

30. The method of claim 1, wherein the test nucleic acid is amplified using random primers.

31. The method of claim 1, wherein the test nucleic acid is amplified using a phi29-like enzyme.

32. The method of claim 7, further comprising identifying a probe sequence on the array wherein a ratio of a first complex comprising the probe sequence to a second complex comprising the probe sequence is different from one and including the probe sequence in the probe set.

33. The method of claim 1, wherein the probe set comprises probe sequences from a plurality of chromosomes of an organism's genome.

34. The method of claim 9, wherein the organism is a human.

35. The method of claim 9, wherein the probe set comprises probe sequences from each chromosome of an organism's genome.

36. The method of claim 1, wherein the probe sequence in the probe set comprises an oncogene.

37. The method of claim 1, wherein the probe sequence in the probe set comprises a tumor suppressor gene.

38. The method of claim 1, wherein the test genomic sample is from a biopsy.

39. The method of claim 1, wherein the test genomic sample is from a tumor.

40. The method of claim 1, wherein the test genomic sample is from fetal cells.

41. The method of claim 1, wherein the test nucleic acid comprises genomic DNA.

42. The method of claim 1, wherein the reference nucleic acid is from a healthy patient or a patient known to have a diploid complement of a reference sequence.

43. The method of claim 1, wherein the reference nucleic acid comprises nucleic acid present at a known copy number.

44. A kit comprising an array comprising a plurality of probe sequences, and a probe set comprising a molecule comprising a probe sequence corresponding to a probe sequence on the array and a sequence complementary to a branched nucleic acid.

45. A method comprising:
performing an enzyme-based amplification method on a sample comprising nucleic acids to obtain amplified nucleic acids;
obtaining a first and second portion of sample comprising the amplified nucleic acids;
contacting amplified nucleic acids in the first portion to a probe set comprising a multi-labeled probe nucleic acid molecule comprising a probe sequence, wherein the multi-labeled probe nucleic acid molecule comprises a plurality of labels; and
comparing a level of nucleic acids complementary to the probe sequence in the second portion to a level of nucleic acids bound to the multi-labeled probe nucleic acid molecule in the first portion.

46. The method of claim 45, wherein the nucleic acids in the sample comprise genomic DNA and the enzyme-based amplification method comprises a whole genome amplification method.

47. The method of claim 45, comprising contacting nucleic acids with random primers in the presence of nucleotides and a polymerase.

48. The method of claim 46, wherein the polymerase is a phi29-like polymerase.

49. The method of claim 45, wherein the multi-labeled probe nucleic acid molecule comprises a multimeric nucleic acid molecule comprising a plurality of repeating sequence units and a probe sequence or a complement of a probe sequence.

50. The method of claim 45, further comprising obtaining a portion of the sample prior to amplification and contacting unamplified nucleic acids in the portion to a probe set comprising a multi-labeled probe nucleic acid molecule comprising a probe sequence, wherein the multi-labeled probe nucleic acid molecule comprises a plurality of labels.

51. The method of claim 50, further comprising comparing a level of nucleic acids complementary to the probe set in the second portion to a level of nucleic acids in the portion of the sample comprising unamplified nucleic acids bound to the probe sequence in the probe set.

52. The method of claim 45, wherein the probe set comprises at least about 5 different probe sequences.

53. The method of claim 45, wherein the probe set comprises at least about 10 different probe sequences.

54. The method of claim 45, wherein the probe set sequences comprise a plurality of different probe sequences.

55. The method of claim 45, wherein the comparing is used to assess an amount of bias in the enzyme-based amplification method.

56. The method of claim 55, wherein the probe set comprises a plurality of different probe sequences and bias is assessed by determining the numbers of different probe sequences in the probe set that binds to the portion of amplified nucleic acids.

57. The method of claim 51, wherein the probe set comprises a plurality of different probe sequences and bias in the enzyme-based amplification procedure is assessed by determining the numbers of different probe sequences that binds to the portion of unamplified nucleic acids compared to numbers of different probe sequences that bind to the first portion of amplified nucleic acids.

58. The method of claim 45, wherein the probe set comprises a plurality of different probe sequences and the relative proportions of bound probes in the first portion of sample comprising amplified sequences is compared to the relative proportions of bound probes in a portion of sample comprising unamplified sequences.

59. The method of claim 45, further comprising labeling amplified sequences in a primer extension reaction by providing either or both labeled primers and nucleotides, and comparing the amount of sequences complementary to sequences of probes in the probe set to the relative proportions of bound probes in the first portion of sample comprising amplified sequences and/or in a portion of sample comprising unamplified sequences.

60. A kit comprising a probe set comprising a multi-label nucleic acid molecule and a polymerase.

61. The kit of claim 60, wherein the polymerase comprises a phi29-like enzyme.

62. The kit comprising a probe set comprising a multi-label nucleic acid molecule and random primers.

63. The kit of claim 60, wherein the kit further comprises random primers.

64. The kit of claim 62, wherein the kit further comprises a phi29-like enzyme.

65. The kit of claim 60 comprising a multi-well plate comprising capture oligonucleotides stably associated with at least a portion of one of the wells.

66. The kit of claim 60, wherein the kit further comprises a phi29-like enzyme.

67. The kit of claim 60 comprising a multi-well plate comprising an array of nucleic acid probes in at least one well.

68. The kit of claim 65, wherein the multi-well plate further comprises at least one array of nucleic acid probes in at least one well.

69. The kit of claim 60, wherein the probe set comprises a plurality of multi-labeled probe nucleic acid molecules, each member of the plurality comprising a different probe sequence.

70. The kit of claim 69, wherein each member of the plurality is provided in a different container or in different wells of a multi-welled container.
